(19) United States
(12) Patent Application Publication
(10) Pub. No.: US 2023/0395215 A1
(43) Pub. Date: Dec. 7, 2023

(54) SCALABLE FRAMEWORK FOR DIGITAL MESH

(71) Applicant: Evernorth Strategic Development, Inc., St. Louis, MO (US)

(72) Inventors: Andrew Long, Austin, TX (US); Christina M. Vallery, Eastsound, WA (US); Chris Haddad, Cocoa Beach, FL (US); Brian S. Mitchell, St. Louis, MO (US)

(21) Appl. No.: 18/205,249

(22) Filed: Jun. 2, 2023

Related U.S. Application Data
(60) Provisional application No. 63/348,385, filed on Jun. 2, 2022.

(51) Int. Cl. G16H 10/60; G16H 50/20

(52) U.S. Cl. CPC G16H 10/60; A61B 5/0022; G16H 50/20

(57) ABSTRACT

Methods and systems for providing a scalable framework for a digital mesh are provided. The methods and systems perform operations comprising: receiving patient information associated with a patient; receiving, via a graphical user interface, a request for health services for the patient; in response to receiving the request for health services, applying a model to the patient information to select a first type of service of care from a plurality of types of service of care; accessing a mesh application service architecture (MASA) to route traffic comprising a portion of the patient information to a server associated with the first type of service of care; and generating, for display within the graphical user interface, health service information received from the server associated with the first type of service of care.

### CROSS-REFERENCE TO RELATED APPLICATION

This application is a non-provisional of and claims priority to U.S. Provisional Application No. 63/348,385, filed Jun. 2, 2022, which is hereby incorporated by reference herein in its entirety.

### BACKGROUND

Patients seek medical treatment in a variety of different ways. Some patients desire medical treatment in person by visiting a live medical professional. Other patients prefer to receive medical treatment virtually. Different systems are provided to access medical treatments based on the preferences of the patients.

### BRIEF DESCRIPTION OF THE DRAWINGS

FIG. 1A is a block diagram of an example digital mesh platform, according to some examples.

FIG. 1B is a block diagram of an example digital mesh platform, according to some examples.

FIG. 2 is an example database that may be deployed within the system of FIG. 1, according to some examples.

FIG. 3 is a block diagram of an example service of care type selection system that may be deployed within the system of FIG. 1, according to some examples.

FIGS. 4 and 5 are example user interfaces of the digital mesh platform, according to examples.

FIG. 6 is a flowchart illustrating example operations of the digital mesh platform, according to examples.

FIG. 7 is a block diagram illustrating an example software architecture, which may be used in conjunction with various hardware architectures herein described.

FIG. 8 is a block diagram illustrating components of a machine, according to some examples.

FIG. 9 is a functional block diagram of an example neural network that can be used for the inference engine or other functions (e.g., engines) as described herein to produce a predictive model.

### DETAILED DESCRIPTION

Example methods and systems for a digital mesh platform are provided. Specifically, the methods and systems provide recommendations for different type of service of care based on the needs, preferences and medical information associated with the patients. In the following description, for purposes of explanation, numerous specific details are set forth in order to provide a thorough understanding of example embodiments. It will be evident, however, to one of ordinary skill in the art that the disclosed examples may be practiced without these specific details.

Patients seek medical treatment in a variety of different ways. Some patients desire medical treatment in person by visiting a live medical professional. Other patients prefer to receive medical treatment virtually. Different systems are provided to access medical treatments based on the preferences of the patients. In order to access treatment of a particular type, a patient typically needs to launch a specific application associated with the desired treatment. The patient needs to navigate many pages of information to input their medical information, login and preferences to schedule and/or receive medical treatment of the particular type. If the patient, at a later time, seeks other types of service of care or medical treatment, a separate different application may need to be launched and accessed. Again information needs to be reinput to receive and/or schedule treatment associated with the other type of service of care. Typical systems fail to provide a single application that aggregates all of the different types of service of care that are available into a single user interface. As a result, patients are burdened with having to navigate multiple pages of information and repeatedly inputting their information to receive treatment. This wastes a great deal of time and resources that can be devoted to other tasks.

The disclosed examples provide systems and methods to orchestrate and aggregate multiple types of services of care into a single application or user interface. Particularly, the disclosed techniques provide a scalable framework for a digital mesh. The disclosed techniques receive patient information associated with a patient and receiving, via a graphical user interface, a request for health services for the patient. The disclosed techniques, in response to receiving the request for health services, apply a model to the patient information to select a first type of service of care from a plurality of types of service of care. The disclosed techniques access a mesh application service architecture (MASA) to route traffic that includes a portion of the patient information to a server associated with the first type of service of care. The disclosed techniques generate, for display within the graphical user interface, health service information received from the server associated with the first type of service of care.

In this way, the disclosed examples improve the overall process of providing medical care and specifically improves the efficiency at which service of care of different types are selected to be provided to a patient. Specifically, the disclosed techniques enable a patient to access a variety of different types of services of care and receive recommendations for certain types of services of care quickly and efficiently through a single application or user interface. As a result, a great deal of time is saved and the user need not have to navigate through a multitude of pages of information to seek medical treatment or repeatedly input their medical information into user interfaces specific to each type of service of care. This saves time and reduces the amount of resources needed to accomplish a task.

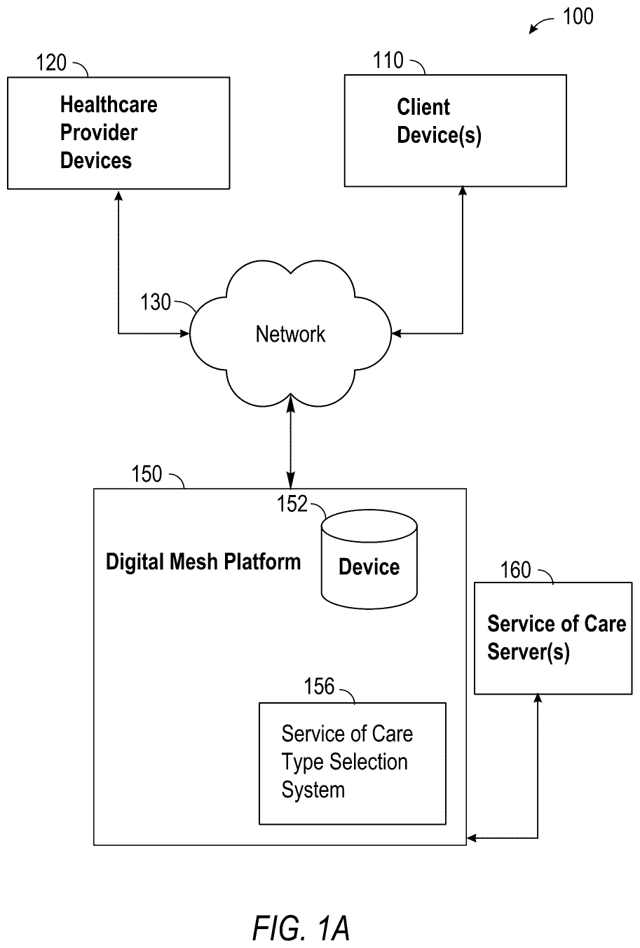

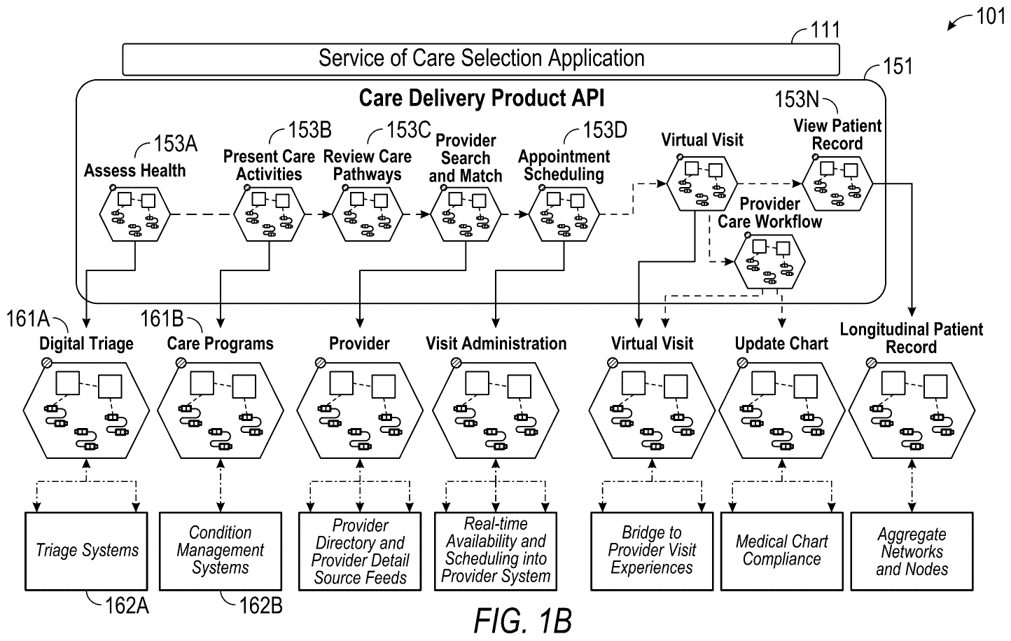

FIG. 1A is a block diagram showing an example digital mesh system 100 according to various exemplary embodiments. The digital mesh system 100 includes one or more client devices 110, one or more healthcare provider devices 120, a digital mesh platform 150, and one or more service of care servers 160 that are communicatively coupled over a network 130 (e.g., Internet, telephony network). Each of the one or more service of care servers 160 hosts a different type of service of care platform or application resources. For example, a first server of the one or more service of care servers 160 provides access to resources associated with in-person service of care medical services and a second server of the one or more service of care servers 160 provides access to resources associated with virtual service of care medical services. Other servers (not shown) provide a variety of other services of care, such as a healthcare chatbot system that implements a large language model (LLM) artificial intelligence system, an appointment scheduling platform, and so forth. These additional types of services of care which are implemented and provided using respective servers are shown and described in detail in connection with FIG. 1B.

As used herein, the term “client device” may refer to any machine that interfaces to a communications network (such as network 130) to access the digital mesh platform 150. The client device 110 may be, but is not limited to, a mobile phone, desktop computer, laptop, portable digital assistants (PDAs), smart phones, a wearable device (e.g., a smart watch), augmented reality (AR) device, virtual reality (VR) device, tablets, ultrabooks, netbooks, laptops, multi-processor systems, microprocessor-based or programmable consumer electronics, game consoles, set-top boxes, an interactive voice response system, or any other communication device that a user may use to access a network or the digital mesh platform 150 via a specific engagement channel.

In some cases, the digital mesh platform 150 is accessible over a global communication system, e.g., the Internet or world wide web. In such instances, the digital mesh platform 150 hosts a website that is accessible to the client devices 110. Upon accessing the website, the client devices 110 provide secure login credentials, which are used to access a profile associated with the login credentials. One or more user interfaces associated with the digital mesh platform 150 are provided over the Internet via the website to the client devices 110.

Healthcare provider devices 120 can include the same or similar functionality as client devices 110 for accessing the digital mesh platform 150. In some cases, the healthcare provider devices 120 are used by “internal” users. Internal users are personnel, such as physicians, clinicians, healthcare providers, health-related coaches pharmacy benefit manager (PBM) operators, pharmacists, specialty pharmacy operators or pharmacists, or the like that are associated with or employed by an organization that provides the digital mesh platform 150. In some cases, the healthcare provider devices 120 are used by “external” users. External users are personnel, such as physicians, clinicians, and health-related coaches that are associated with or employed by a different (external) organization than that which provides the digital mesh platform 150.

The healthcare provider devices 120, in some cases, are used to access a respective server of the service of care servers 160. For example, a first group of healthcare provider devices 120 can be associated with medical personnel that provide in-person medical services. In such cases, the first group of the healthcare provider devices 120 can access a first set of the service of care servers 160 that provide in-person medical services resources or implement in- person medical services applications. A second group of healthcare provider devices 120 can be associated with medical personnel that provide virtual medical services. In such cases, the second group of the healthcare provider devices 120 can access a second set of the service of care servers 160 that provide virtual medical services resources or implement virtual medical services applications.

The healthcare provider devices 120, when used by internal or external users, to access the digital mesh platform 150 can view many records associated with many different patients (or users associated with client devices 110). Different levels of authorization can be associated with different internal and different external users to control which records the internal and external users have access. In some instances, only records associated with those patients to which a given internal or external user is referred, are made accessible and available to the given internal or external user device. Sometimes, a first internal or external user can refer a patient or records associated with the patient to a second internal or external user. In such circumstances, the second internal or external user becomes automatically authorized to access and view the patient's records that were referred by the first internal or external user.

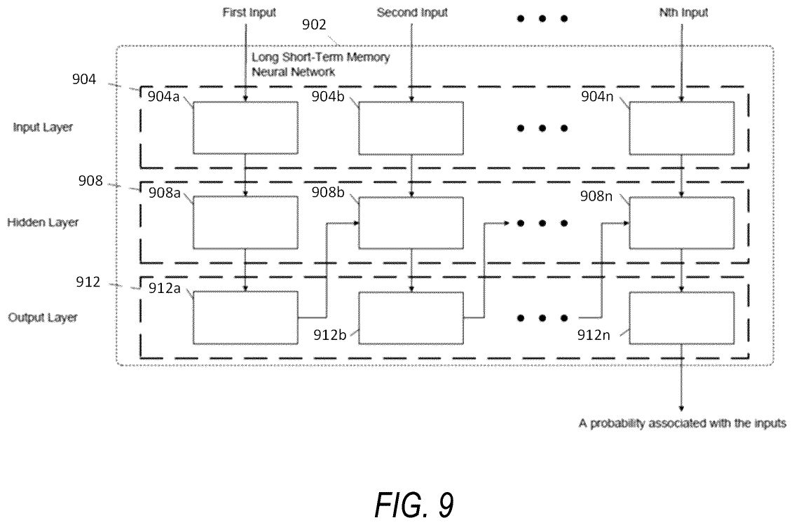

In some examples, the digital mesh platform 150 (and specifically the service of care type selection system 156) can implement a machine learning technique or machine learning model, such as a neural network (discussed below in connection with FIG. 9). The machine learning technique can be trained to establish a relationship between a plurality of training patient information features and types of service of care. In an example, the machine learning technique can be trained by obtaining a batch of training data that includes a first set of the plurality of training patient information features associated with a given type of service of care. The first set of the plurality of training patient information features is processed by the machine learning technique to generate an estimated type of service of care. A loss can be computed based on a deviation between the estimated type of service of care and the given type of service of care associated with the first set of the plurality of training patient information features. Parameters of the machine learning technique can then be updated based on the computed loss.

These training operations can be repeated for multiple batches of training data and/or until a stopping criterion is reached. For example, a second batch of training data that includes a second set of the plurality of training patient information features associated with a second given type of service of care can be obtained. The second set of the plurality of training patient information features is processed by the machine learning model to generate a second estimated type of service of care and a loss can be computed based on a deviation between the second estimated type of service of care and the second given type of service of care associated with the second set of the plurality of training patient information features. The parameters of the machine learning model are again update based on the computed loss.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The healthcare provider devices 120 can be used to access pharmacy claims, medical data (e.g., medical information 230 stored in database 152), laboratory data and the like for one or more patients that the healthcare provider devices 120 are authorized to view. This patient information 210 can be maintained in a database 152 by the digital mesh platform 150 or in a third-party database accessible to the digital mesh platform 150 and/or the healthcare provider devices 120.

In some examples, the client devices 110 and the digital mesh platform 150 can be communicatively coupled via an audio call (e.g., VoIP, Public Switched Telephone Network, cellular communication network, etc.) or via electronic messages (e.g., online chat, instant messaging, text messaging, email, and the like). While FIG. 1 illustrates a single client device 110 and a single healthcare provider device 120, it is understood that a plurality of such devices can be included in the system 100 in other embodiments. As used herein, the term “client device” may refer to any machine that interfaces to a communications network (such as network 130) to obtain resources from one or more server systems or other client devices through an engagement channel.

The digital mesh platform 150 can be a human agent or an automated agent, e.g., on behalf of an organization. The automated agent can be associated with a medical group that includes the member. The automated agent can be an interactive voice response (IVR), a virtual online assistant, or a chatbot provided on a website. In cases of a chatbot, the chatbot can be implemented by a particular healthcare chatbot server or system that is coupled to the digital mesh platform 150. The healthcare chatbot server can implement an LLM artificial intelligence system that is specifically trained to respond to medical related prompts. LLM artificial intelligence system can be trained to establish a relationship between a prompt (e.g., image, text, audio, video, and so forth) and visual and/or audible outputs corresponding to various types of service of care. In an example, the LLM can be trained by obtaining a batch of training data that includes a first set of prompts representing a set of patient information features associated with a given output corresponding to one or more types of service of care. The first set of the prompts is processed by the LLM to generate an estimated output. A loss can be computed based on a deviation between the estimated output and the given output associated with the first set of the prompts. Parameters of the LLM can then be updated based on the computed loss. These training operations can be repeated for multiple batches of training data and/or until a stopping criterion is reached.

During a communication session between the user and the agent, the digital mesh platform 150 identifies the member using initial context data (e.g., the phone number the member is calling from, the website login information inputted, automatic number identification (ANI), etc.) and retrieves the data on the member (e.g., member account information, name, address, insurance information, information on spouse and dependents, etc.) to be presented on the client device 110.

In some examples, the digital mesh platform 150 includes a service of care type selection system 156. In some examples, the service of care type selection system 156 processes medical information input by a patient via the client device 110. For example, the client device 110 can present a graphical user interface to the patient. The graphical user interface can receive input from the patient that provides a variety of medical information, such as patient health information, patient demographic information, patient in-network insurance coverage, patient out-of-network insurance coverage, patient location, and/or one or more treatment preferences. In some cases, the patient health information can be input by receiving selection from the patient of one or more checkboxes from a user interface, each associated with a different medical condition, such as high blood pressure, diabetes, obesity, demographics, and so forth. The patient in-network insurance coverage and out-of-network insurance coverage be input by receiving selection from the patient of one or more checkboxes from a user interface, each associated with a different health insurance carrier. Based on the selected health insurance carrier and health plan information input by the patient, the patient in-network and out-of-network insurance coverages can be automatically determined and retrieved. The patient location can be automatically determined by accessing location information (e.g., GPS coordinates) from the client device 110 and/or by requesting a residential address from the patient. The treatment preferences can be input by receiving selection from the patient specifying how the patient likes to receive treatment, such as in-person, virtually, in a structured format, in aspirational format (e.g., a format that lacks structure), and so forth.

The service of care type selection system 156 can store one or more rules for selecting a service of care type. For example, a first set of one or more rules can be associated with a first type of service of care and a second set of one or more rules can be associated with a second type of service of care. In some cases, a default type of service of care can be specified (e.g., virtual care) if no rules are satisfied by medical information received and/or stored for a given patient. The service of care type selection system 156 receives the medical information from the patient via the client device 110. The service of care type selection system 156 applies the one or more rules to the medical information to determine which of the sets of rules are satisfied. The service of care type selection system 156 can select a first type of service of care in response to determining that the first set of the one or more rules are met or satisfied by the medical information. The service of care type selection system 156 can select a second type of service of care in response to determining that the second set of the one or more rules are met or satisfied by the medical information.

For example, the medical information can specify a preference for in-person medical care. In such cases, the service of care type selection system 156 can determine that the first set of rules corresponding to the in-person medical care type of service are satisfied by the medical information. As another example, the medical information can specify in-network medical coverage in the patient's insurance plan that requires virtual visits to be performed or conducted. In such cases, the service of care type selection system 156 can determine that the first set of rules corresponding to the in-person medical care type of service fail to be satisfied by the medical information and that the second set of rules corresponding to virtual visits or care are satisfied. In such cases, the service of care type selection system 156 selects the second type of service of care corresponding to virtual visits. The selected service of care type is then recommended to the patient and/or used to retrieve and/or schedule medical services for the patient. As another example, the medical information can specify in-network medical coverage in the patient's insurance plan that identifies a first type of health services personnel including doctors for treating the patient rather than a second type of health services personnel including nurses, nurse practitioners, or pharmacists. In such cases, the service of care type selection system 156 can determine that a third set of rules corresponding to the first type of health services personnel is satisfied by the medical information. In such cases, the service of care type selection system 156 selects the first type of health services personnel and routes traffic to medical personnel devices and/or servers corresponding to the first type of health services personnel.

In some examples, the rules are presented or made available to the user of the client device 110. The user can update, review, and/or modify any of the rules used to select a given type of service of care. In some examples, the client device 110 can receive input from a user that defines a new rule for selecting the given type of service of care. This new rule can be reviewed by a system moderator and can then be added to the list of rules used by the care type selection system 156 to select a type of service of care based on patient information it receives. In some examples, interactions made between a user and a given type of service of care is fed back to the care type selection system 156 and optionally to another type of service of care. This provides a coherent experience for the user, such as if the user initially interacts with one type of service of care and then decides to transition to another type of service of care. The experience for the user is seamless and updated information is not needed to be collected from the user when the user is connected to the other type of service of care. In some examples, an interaction performed by a user on one type of service of care can trigger activation and routing of traffic to a second type of service of care. This can result in automatically navigating the user to a user interface of the second type of service of care in response to a trigger or operations or interactions the user performs with the initially selected type of service of care.

For example, a user can interact with a healthcare chatbot type of service of care and can ask certain medical questions to the healthcare chatbot. The healthcare chatbot can determine that one or more questions being asked by the user are incompatible with medical capabilities of the healthcare chatbot. In such cases, the healthcare chatbot can automatically send a trigger or message a virtual visit type of service of care. The trigger provided to the virtual visit type of service of care can be routed through the care type selection system 156. The trigger can include at least some of the one or more questions that were asked by the user. The care type selection system 156 can process insurance related information for the user to activate and engage with the virtual visit type of service of care. The care type selection system 156 can then connect the user directly with virtual visit type of service of care, such as through the same user interface used by the user to engage with the healthcare chatbot. The virtual visit type of service of care can then respond to the one or more questions that were asked by the user without the user having to reinput the one or more questions. This is all done seamlessly and with minimal or no user intervention.

In some examples, the care type selection system 156 can determine that the medical information received from the user is insufficient to select a particular type of service of care. In such cases, the care type selection system 156 can process the medical information to generate one or more follow-up questions. The care type selection system 156 can prompt the client device 110 to provide a response to the one or more follow-up questions presented in a notification on the client device 110 to update the medical information. The care type selection system 156 can then process the follow-up questions and select a suitable type of service of care that is associated with one or more rules that match the updated medical information. The care type selection system 156 can route traffic including the updated medical information to the server associated with the selected type of service of care. The care type selection system 156 can then manage and present an interface associated with the server on the client device 110 without requiring the user to navigate to a separate user interface associated with the server for the selected type of service of care.

In some examples, a new service of care type can be added to the digital mesh platform 150 for access by the same user interface on the client device 110. To add the new service of care type, a server associated with the service of care type is added to the one or more service of care servers 160 along with an API associated with the new service of care type. A set of rules associated with the new service of care type is provided and added to the service of care type selection system 156. Now, when a request for medical services is received from a client device 110, the medical information can be processed by all of the rules including the new set of rules. If the new set of rules is satisfied, the API associated with the new service of care type is used to communicate and exchange data with the one or more service of care servers 160 and to present information corresponding to the new service of care type in the graphical user interface on the client device 110. In this way, the same user interface can be utilized to access a variety of different service of care types without requiring the user to re-input medical information each time the user seeks or requests a medical service. The digital mesh platform 150 can intelligently identify the most suitable service of care type to recommend to the patient based on the previously input patient information and can navigate the user automatically to the appropriate user interface within the same application that provides access to a variety of types of service of care.

In some examples, to add the new service of care type to the digital mesh platform 150, the digital mesh platform 150 can process data associated with the server for the new service of care type. The digital mesh platform 150 can analyze the format of the data and can determine whether the format of the data corresponds to a format of the data stored and processed by the digital mesh platform 150. In response to determining that the data is formatted in a way that is incompatible with the digital mesh platform 150, the digital mesh platform 150 can process the data to normalize the data prior to adding the server or a link to the server in the digital mesh platform 150. In this way, all of the data received from various servers coupled to the digital mesh platform 150, is standardized and normalized prior to being aggregated. This standardized format allows the data to be presented in a coherent way on the user interface of the client device 110 provided by the digital mesh platform 150.

In some cases, prior to adding the new service of care type, one or more assessment criteria are analyzed in an automated manner. The one or more assessment criteria can include discoverability and review of the user interfaces of the new service of care type, availability of real-time interactions and communication sessions, extensibility, composability and orchestration, availability of analytics, security, resilience, and/or observability. Extensibility represents the data model used by the new service of care type server and whether data resource dependencies are hard coded or flexible. Composability and orchestration represents the ability to route traffic to back-end providers based on consumer configuration and ecosystem relationships and a proven ability to aggregate data and interactions across more than one back-end service or dataset. Security represents whether consent access policies are in place in the new service of care type and whether authentication protocols are in place that conform to secure authentication protocols of the digital mesh platform 150.

The new service of care type can be scored based on these one or more assessment criteria. If the score transgresses a specified threshold, the new service of care type can be integrated and added to the digital mesh platform 150. If not, the service of care type operator is informed about which of the one or more assessment criteria failed to be satisfied or ranked below a specified value for purposes of improving the integrability of the service of care type.

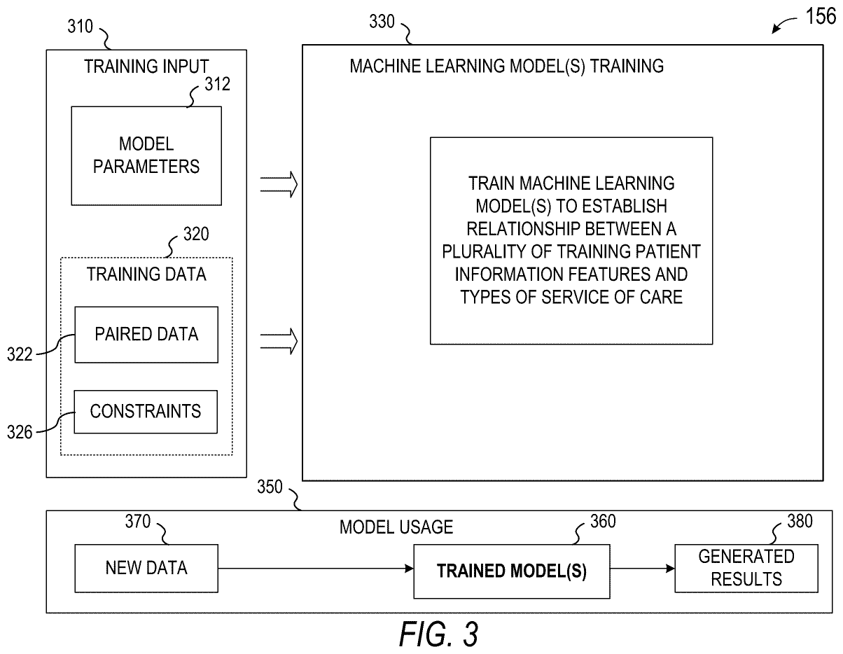

In some examples, as explained in more detail in connection with FIG. 3, the service of care type selection system 156 is trained based on training patient information features (e.g., patient health information, patient demographic information, patient in-network insurance coverage, patient out-of-network insurance coverage, patient location, or one or more treatment preferences) and their corresponding ground-truth types of service of care. The service of care type selection system 156 is trained to predict type of service of care for a given set of patient information features. The prediction can be used to select a type of service of care to recommend to a patient.

The type of service of care that is recommended or selected (by way of the rules and/or the prediction provided by the machine learning model) is then used to select an Application Programming Interface (API) from a variety of APIs that is associated with the recommended service of care type. The select API is then used to communicate with a particular one of the service of care servers 160. Specifically, the API is used to call a function (perform a function call) that takes as input at least a portion of medical information received and/or stored in association with a patient. A query is generated to rout the portion of the medical information to the particular one of the service of care servers 160 with a request for medical information. The medical information received from the one of the service of care servers 160 is then presented in a graphical user interface to the patient. For example, a list of healthcare professionals (providers) that provide the recommended service of care type is received from the one of the service of care servers 160. The list of healthcare professionals is then filtered and/or sorted to select a subset of the healthcare professions to present to the patient on the client device 110. The client device 110 can receive input from the patient selecting a given one of the healthcare professionals to conduct a live virtual visit or schedule an in-person appointment.

FIG. 1B is a block diagram 101 of an example digital mesh platform 150, according to some examples. The block diagram 101 includes a service of care selection application 111, a list of various types of service of care service 151 available by the digital mesh platform 150, various servers 161A and 161B associated with each service of care type, and various backend systems 162A and 162B coupled to the various servers 161A and 161B. In some examples, the care selection application 111 can be accessed by a user through the client device 110. The client device 110 can implement the care selection application 111 that is coupled to the digital mesh platform 150. The care selection application 111 can provide a unified interface for the user to browse and interact with a variety of types of service of care.

In some examples, medical information is collected from the user via the care selection application 111. The medical information is provided to the care service 151 which can be a component of the digital mesh platform 150. The care service 151 can process the medical information and, based on one or more rules and/or a model, select a particular service of care type that matches the medical information and/or request received from the care selection application 111. In some cases, the care service 151 can determine that the medical information includes a request to assess health of a patient. In such cases, the care service 151 selects a first type of service of care 153A. The first type of service of care 153A can correspond to a health assessment service. This first type of service of care 153A can receive the patient information and can communicate through an API with a data model of a corresponding server 161A. The server 161A can implement services associated with health assessment and can provide a user interface on the care selection application 111 that represents functionality associated with health assessment. In some cases, the server 161A can communicate with one or more backend systems 162A that are or are not coupled to the digital mesh platform 150. The server 161A can use the one or more backend systems 162A to respond and/or generate the user interface presented to the care selection application 111.

In some cases, the care service 151 can determine that the medical information includes a request to view a list of available care activities or programs. In such cases, the care service 151 selects a second type of service of care 153B. The second type of service of care 153B can correspond to a list of different care programs. This second type of service of care 153B can receive the patient information and can communicate through an API with a data model of a corresponding server 161B. The server 161B can implement services associated with condition management systems and can provide a user interface on the care selection application 111 that represents functionality associated with care programs suitable for the medical information. In some cases, the server 161B can communicate with one or more backend systems 162B that are or are not coupled to the digital mesh platform 150. The server 161B can use the one or more backend systems 162B to respond and/or generate the user interface presented to the care selection application 111. Similarly, the care service 151 can select other types of service of care, such as a review of care pathways 153C, appointment scheduling 153D, and/or access to patient records 153N. Each of these different types of service of care can communicate with each other, such as via the digital mesh platform 150 and can be associated with respective servers 161 that implement the functionality they provide. Each server and/or its corresponding backend system can be configured to be decoupled from the digital mesh platform 150 and can be accessed by a user independently of the digital mesh platform 150.

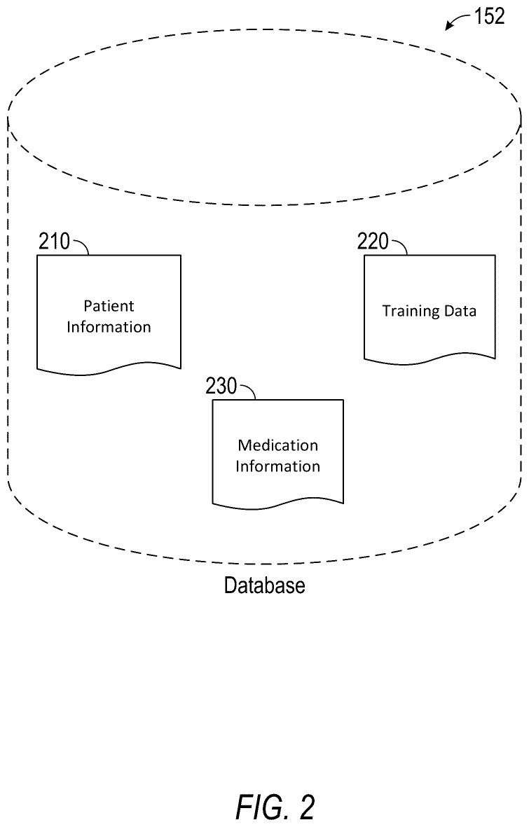

FIG. 2 is an example database 152 that may be deployed within the system of FIG. 1, according to some embodiments. The database 152 includes patient information 210, medication information 230, and training data 220. The patient information 210 can be generated by the digital mesh platform 150. For example, the digital mesh platform 150 can access one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 110 and so forth. The digital mesh platform 150 can collect this information from the patient records and generates a patient features vector that includes this information.

The medication information 230 stores various medication related information (e.g., prescriptions, size of the medication or pills, compatible forms of dispensing information, temperature control information, mixing exclusion information, and so forth) for various medications. The medication information 230 can also store various information about medical personnel and which types of services they provide along with their respective geographical locations. The medication information 230 is used to populate a user interface that is associated with a particular service of care type that is selected/recommended to the patient and that is presented on the client device 110.

The training data 220 includes training sets of the plurality of training patient information features associated with different types of service of care. The training data 220 is used to train a machine learning model implemented by the service of care type selection system 156 to generate estimates of one or more service of care types to recommend to a patient. For example, the training data 220 can be built over time by identifying a first set of the plurality of training patient information features that are associated with a given type of service of care and by identifying a second set of the plurality of training patient information features that are associated with a second given type of service of care. The training data 220 can also store preference information for various categories or groups of individuals, such as by age group, indicating the types of medication regimens which are preferred, treatment structure types, physician types, and so forth.

FIG. 3 is a block diagram of an example service of care type selection system 156 that may be deployed within the system of FIG. 1, according to some embodiments. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these parameters 312 are adapted based on the input-output training pairs of the training data sets 322. After the parameters 312 are adapted (after training), the parameters are used by trained models 360 to implement the trained machine learning models on a new set of data 370.

Training data 320 includes constraints 326 which may define the constraints of a given patient information features. The paired training data sets 322 may include sets of input-output pairs, such as a pairs of a plurality of training patient information features and types of service of care associated with the training patient information features. Some components of training input 310 may be stored separately at a different off-site facility or facilities than other components.

Machine learning model(s) training 330 trains one or more machine learning techniques based on the sets of input-output pairs of paired training data sets 322. For example, the model training 330 may train the machine learning (ML) model parameters 312 by minimizing a loss function based on one or more ground-truth type of service of care. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the ML model can be applied to a training plurality of patient information features to estimate or generate a prediction of a type of service of care. In some implementations, a derivative of a loss function is computed based on a comparison of the estimated type of service of care and the ground truth type of service of care associated with the training patient information features and parameters of the ML model are updated based on the computed derivative of the loss function.

In one example, the ML model receives a batch of training data that includes a first set of the plurality of training patient information features together with a ground-truth type of service of care associated with the first set of the plurality of training patient information features. The ML model generates a feature vector based on the first set of the plurality of training patient information features and generates a prediction of one or more types of service of care. The prediction is compared with the ground truth type of service of care and parameters of the ML model are updated based on the comparison.

The result of minimizing the loss function for multiple sets of training data trains, adapts, or optimizes the model parameters 312 of the corresponding ML models. In this way, the ML model is trained to establish a relationship between a plurality of training patient information features and types of service of care.

After the machine learning model is trained, new data 370, including one or more patient information features are received. The trained machine learning technique may be applied to the new data 370 to generate results 380 including a prediction of a service of care type to recommend. The selection or recommendation made by the service of care type selection system 156 can be represented in a graphical user interface that depicts each of a plurality of different types of service of care.

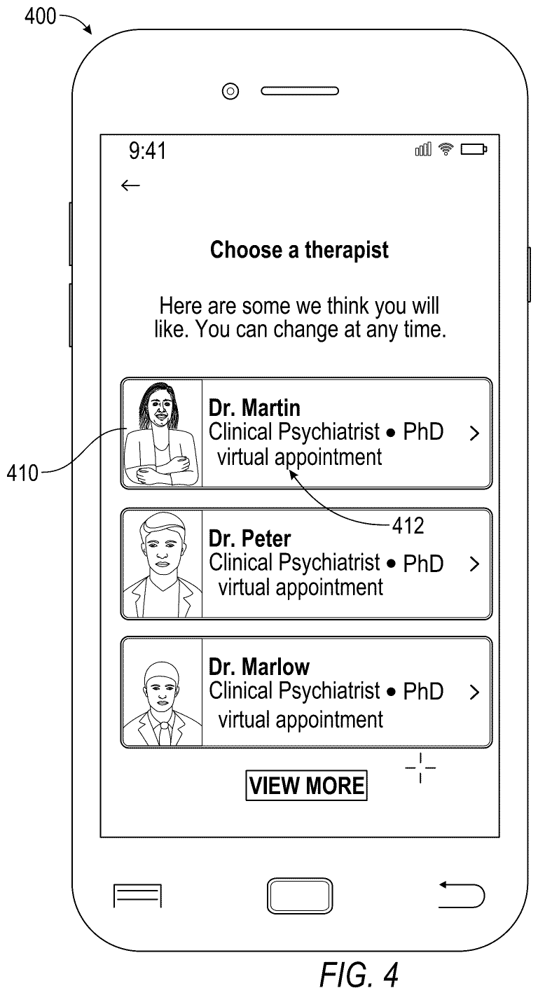

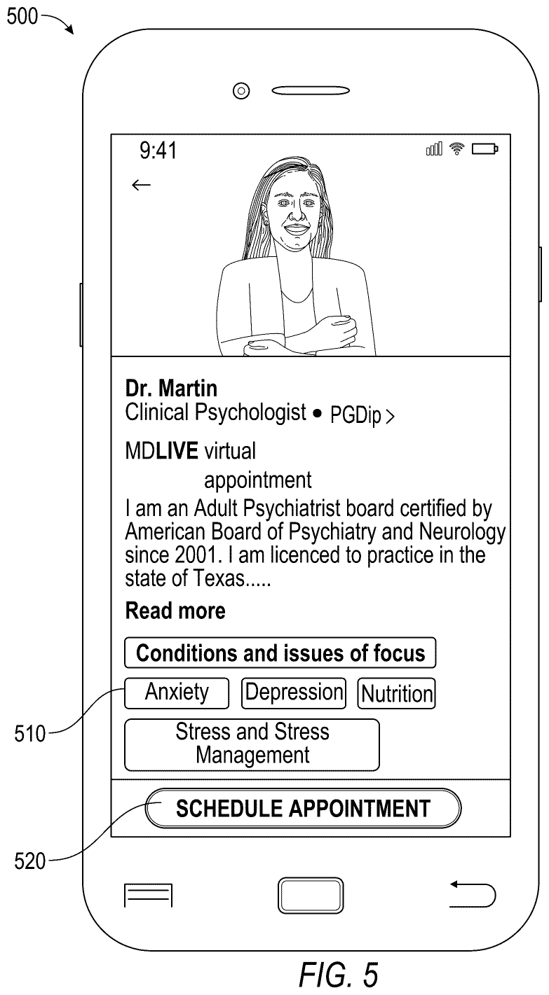

FIGS. 4 and 5 are example user interfaces 400 and 500 of the digital mesh system 100, according to example embodiments. For example, a client device 110 can provide medical information (patient information) associated with a patient to the service of care type selection system 156. To do so, the client device 110 can present a graphical user interface through which the patient inputs various patient information. The patient can also input a request for medical services, such as a request to schedule an appointment, fulfill a prescription, or view a list of medical providers.

In response to receiving, the request from the client device 110, the service of care type selection system 156 can apply a model (e.g., one or more rules and/or a machine learning model) to the medical information received from the client device 110. The service of care type selection system 156 can select a virtual visit type of service of care based on the medical information. In response, the service of care type selection system 156 navigates the user to the user interface 400. The service of care type selection system 156 can retrieve an API associated with the virtual visit and generate a query to a given server associated with the virtual visit.

The server can return a list of medical providers that match the medical information that is provided in the query, including medical professionals within a specified range of a location associated with the client device 110. The service of care type selection system 156 can present the medical providers received from the server in the user interface 400. The medical providers can be filtered and/or sorted to generate a subset of providers based on how closely their qualifications and available medical services match the patient information. In an example, a first medical provider 410 can be presented along with an identifier 412 of the type of service that has been selected by the service of care type selection system 156.

The user interface 400 can receive a selection of the first medical provider 410 and, in response, the user interface 500 is presented to the patient on the client device 110. The user interface 500 includes a list of medical services 510 provided by the first medical provider 410. In some cases, a given medial service can be visually distinguished which has been determined to match the patient information received from the patient via the client device 110. The user interface 500 can include a schedule appointment option 520 to enable the patient to schedule an appointment to obtain medical services from the first medical provider 410.

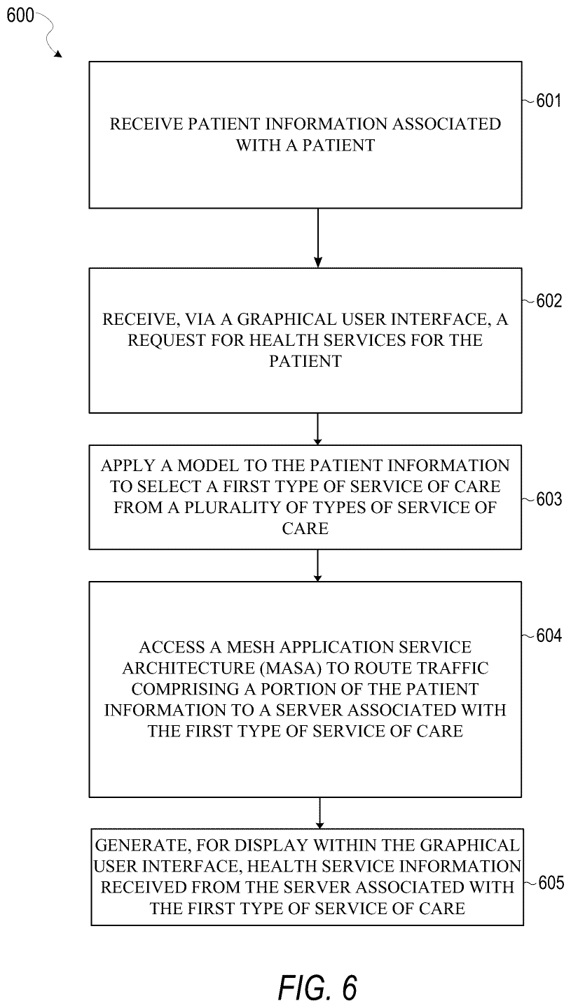

FIG. 6 is a flowchart illustrating example operations of the service of care type selection system in performing process 600, according to example embodiments. The process 600 may be embodied in computer-readable instructions for execution by one or more processors such that the operations of the process 600 may be performed in part or in whole by the functional components of the system 100; accordingly, the process 600 is described below by way of example with reference thereto. However, in other embodiments, at least some of the operations of the process 600 may be deployed on various other hardware configurations. Some or all of the operations of process 600 can be in parallel, out of order, or entirely omitted.

At operation 601, the system 100 receives patient information associated with a patient, as discussed above.

At operation 602, the system 100 receives, via a graphical user interface, a request for health services for the patient, as discussed above.

At operation 603, the system 100 in response to receiving the request for health services, applies a model to the patient information to select a first type of service of care from a plurality of types of service of care, as discussed above.

At operation 604, the system 100 accesses a mesh application service architecture (MASA) to route traffic comprising a portion of the patient information to a server associated with the first type of service of care, as discussed above.

At operation 605, the system 100 generates, for display within the graphical user interface, health service information received from the server associated with the first type of service of care, as discussed above.

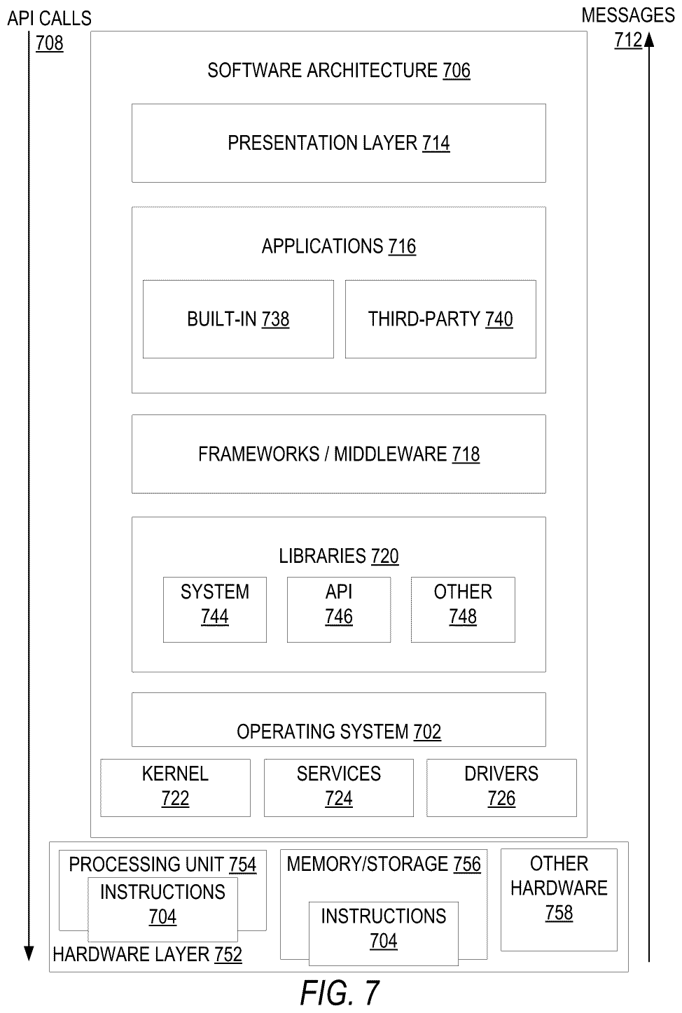

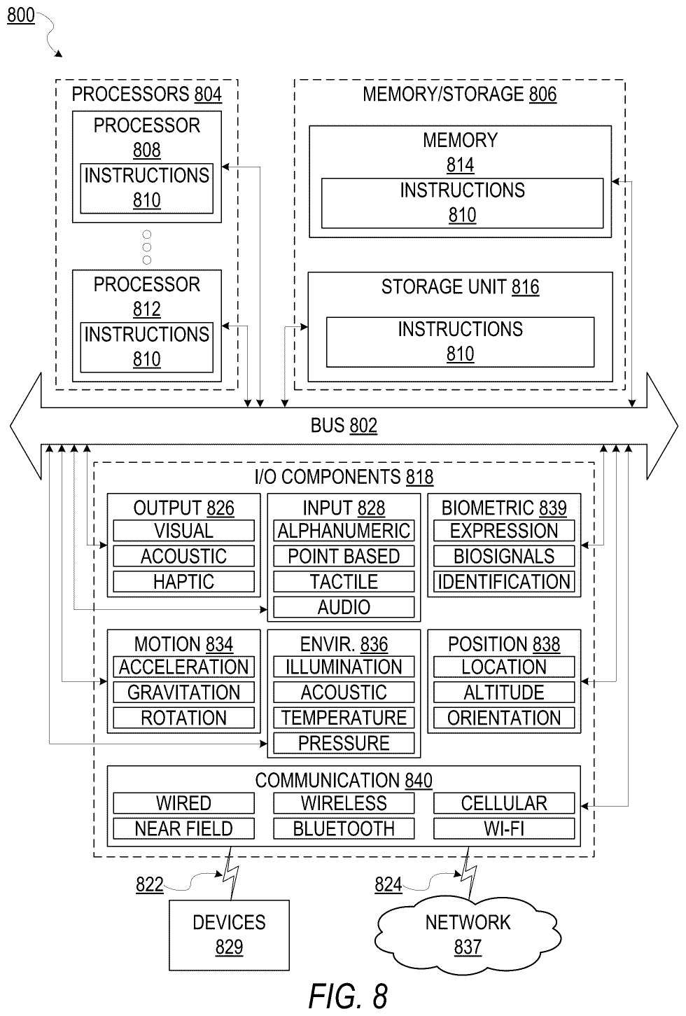

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1. The software architecture 706 can be utilized to apply a machine learning technique or model to generate a prediction of a service of care type to recommend to a patient.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some example embodiments, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process a combination of patient information features with a trained machine learning model to predict a type of service of care to recommend to the patient associated with the patient information features.

As such, the instructions 810 may be used to implement devices or components described herein. The instructions 810 transform the general, non-programmed machine 800 into a particular machine 800 programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

FIG. 9 is a functional block diagram of an example neural network 902 that can be used for the inference engine or other functions (e.g., engines) as described herein to produce a predictive model. The predictive model can identify a type of service of care to recommend for particular patient information. In an example, the neural network 902 can be a LSTM neural network. In an example, the neural network 902 can be a recurrent neural networks (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b . . . 904n. The hidden layer 908 includes neurons 908a, 908b . . . 908n. The output layer 912 includes outputs 912a, 912b . . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training patient information data features may be processed to create the inputs 904a-904n. The neural network 902 may implement a model to produce service of care type prediction or selection for at least one of the patient information data features. More specifically, the inputs 904a-904n can include patient information data features (binary, vectors, factors or the like) stored in the storage device 110. The patient information data features can specify at least one of patient health information, patient demographic information, patient in-network insurance coverage, patient out-of-network insurance coverage, patient location, or one or more treatment preferences.

The patient information data features can be provided to neurons 908a-908n for analysis and connections between the known facts. The neurons 908a-908n, upon finding connections, provides the potential connections as outputs to the output layer 912, which determines a service of care type from many different service of care types.

The neural network 902 can perform any of the above calculations. The output of the neural network 902 can be used to trigger service of care type selection to recommend to a patient in a graphical user interface. For example, the notification can be provided to a PBM, health plan manager, pharmacy, physician, caregiver, and/or a patient.

In some embodiments, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904ais connected to each of neurons 908a, 908b . . . 908n. In various implementations, the provider matching may perform a federated search across multiple sites with multiple APIs. For example, there may not be a single database having all information about all providers, and the multiple APIs may be used to obtain desired information about providers from multiple sources (such as provider websites, provider review and rating sites, databases of provider insurance associations, etc.). In an example, the use of a federated search allows the provider matching system to access new databases by adding an API while the prior APIs can still be used. The inputs from the user device can be parsed, normalized and ranked to generate the APIs. In an example embodiment, a model, e.g., a predictive model as described herein, can be used to perform at least one of the parsing, normalization and ranking of the inputs for a provider match function.

The scalable digital mesh may provide for federated searching capabilities, e.g., by providing a multidimensional search that leverages multiple data sources or categories (e.g., dimensions) in order to improve provider matching with patients, providing relevant health content to a patient device, providing motivation data to a patient device, selecting a treatment care pathway, and the like. For example, provider matching may match providers based on efficiency of providers or how often the providers lead to successful patient outcomes, behavioral or personality factors of providers (e.g., based on reviews of provider mannerisms, whether a provider has a Type A personality, whether a provider has a kind bedside manner, whether the provider has a heart of a teacher, etc.), where the provider is located, what is the provider's specialty or level of expertise (e.g., based on educational background, length of practice, depth of knowledge, etc.), cost of providers, reviews of providers, etc. Social triggers may be obtained by scanning personal profiles of providers, such as by mining via natural language processing (NLP). In various implementations, different weights may be applied to different provider factors based on, e.g., user preferences, etc. These weights may be stored in a table in memory. The table can be accessed applied after the inputs from the patient are parsed and normalized. The weights can rank the inputs for provider matching.

The provider matching application may ask a patient question in an interactive format, in order to narrow down provider matches for the patient. For example, in a behavioral health use case, after a patient logs into an application it may search for all providers in a patient's state (which may be pulled from databases, etc.).

The application may ask questions to the patient about the patient's condition, and use the response information to filter the matched providers. In various implementations, each patient input response may trigger the application to initiate a new API call to pull more information from outside the application, such as searching data sources to identify, e.g., only providers within a specified health insurance coverage plan, only providers with a specified age or gender, etc. In an example, the provider matching may not wait for the entire, complete entry of the patient preference inputs to begin generating APIs to retrieve data from at least one database. The provider matching may parse the preference input as it is entered and begin the searching calls, using e.g., the APIs, before all patient inputs are received. When the returned data is received, then the provider match may rank, e.g., weight, the return data to establish one or more provider data records that match the patient inquiry.

### Glossary:

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

“TIMESTAMP” in this context refers to a sequence of characters or encoded information identifying when a certain event occurred, for example giving date and time of day, sometimes accurate to a small fraction of a second.

Changes and modifications may be made to the disclosed embodiments without departing from the scope of the present disclosure. These and other changes or modifications are intended to be included within the scope of the present disclosure, as expressed in the following claims.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A method comprising:
receiving, by one or more processors, patient information associated with a patient;
receiving, via a graphical user interface, a request for health services for the patient;
in response to receiving the request for health services, applying a model to the patient information to select a first type of service of care from a plurality of types of service of care;
accessing a mesh application service architecture (MASA) to route traffic comprising a portion of the patient information to a server associated with the first type of service of care; and
generating, for display within the graphical user interface, health service information received from the server associated with the first type of service of care.

2. The method of claim 1, further comprising:
obtaining an Application Programming Interface (API) associated with the first type of service;
generating a function call based on the API to communicate the traffic to the server via the MASA; and
generating a query to the server based on the function call.

3. The method of claim 1, wherein the first type of service of care comprises an in-person service of care, wherein a second type of service of care of the plurality of types of service of care comprises a virtual service of care or home visit, wherein a third type of service of care of the plurality of types of service of care comprises a healthcare chatbot service of care, the healthcare chatbot service of care configured as a large language model (LLM) artificial intelligence system.

4. The method of claim 1, wherein the first type of service of care is associated with a first type of health services personnel, wherein a second type of service of care of the plurality of types of service of care is associated with a second type of health services personnel.

5. The method of claim 4, wherein the first type of health services personnel comprises doctors, and wherein the second type of health services personnel comprises nurses, nurse practitioners, or pharmacists.

6. The method of claim 1, wherein the request for health services comprises a request to schedule a health care visit.

7. The method of claim 1, wherein the patient information comprises at least one of patient health information, patient demographic information, patient in-network insurance coverage, patient out-of-network insurance coverage, patient location, or one or more treatment preferences.

8. The method of claim 7, wherein the one or more treatment preferences comprise a preference treatment having a specified structure.

9. The method of claim 7, wherein the one or more treatment preferences comprise a preference for treatment that is aspirational and lacks structure.

10. The method of claim 1, further comprising:
obtaining a list of providers associated with the first type of service of care, each provider on the list being associated with a particular treatment regimen and location; and
selecting a subset of providers from the providers in the list based on the patient information and based on performance information associated with each of the providers in the list of providers.

11. The method of claim 10, wherein the subset of providers is presented as recommended providers for the first type of service in the graphical user interface presented to the patient.

12. The method of claim 1, wherein applying the model comprises:
accessing a list of healthcare specific rules; and
processing the patient information based on the healthcare specific rules to select the first type of service of care from the plurality of types of service of care.

13. The method of claim 1, wherein the model comprises a machine learning model comprising a neural network, the machine learning model trained to establish a relationship between a plurality of training patient information features and types of service of care.

14. The method of claims 13, further comprising training the machine learning model by performing operations comprising:
obtaining a batch of training data comprising a first set of the plurality of training patient information features associated with a given type of service of care;
processing the first set of the plurality of training patient information features by the machine learning model to generate an estimated type of service of care;
computing a loss based on a deviation between the estimated type of service of care and the given type of service of care associated with the first set of the plurality of training patient information features; and
updating parameters of the machine learning model based on the computed loss.

15. The method of claims 14, further comprising training the machine learning model by performing operations comprising:
obtaining a second batch of training data comprising a second set of the plurality of training patient information features associated with a second given type of service of care;
processing the second set of the plurality of training patient information features by the machine learning model to generate a second estimated type of service of care;
computing a loss based on a deviation between the second estimated type of service of care and the second given type of service of care associated with the second set of the plurality of training patient information features; and
updating the parameters of the machine learning model based on the computed loss.

16. The method of claim 1, further comprising:
normalizing data received from a plurality of servers associated with each of the plurality of types of service of care according to parameters of the MASA; and
adding a connection to a respective server of the plurality of servers in response to normalizing the data to orchestrate, aggregate and route the traffic to each respective server, each of the plurality of servers being configured to be decoupled from the MASA.

17. A system comprising:
one or more processors coupled to a memory comprising non-transitory computer instructions that when executed by the one or more processors perform operations comprising:
receiving patient information associated with a patient;
receiving, via a graphical user interface, a request for health services for the patient;
in response to receiving the request for health services, applying a model to the patient information to select a first type of service of care from a plurality of types of service of care;
accessing a mesh application service architecture (MASA) to route traffic comprising a portion of the patient information to a server associated with the first type of service of care; and
generating, for display within the graphical user interface, health service information received from the server associated with the first type of service of care.

18. The system of claim 17, the operations further comprising:
obtaining an Application Programming Interface (API) associated with the first type of service;
generating a function call based on the API to communicate the traffic to the server via the MASA; and
generating a query to the server based on the function call.

19. The system of claim 17, wherein the first type of service of care is associated with a first type of health services personnel, wherein a second type of service of care of the plurality of types of service of care is associated with a second type of health services personnel.

20. A non-transitory computer readable medium comprising non-transitory computer-readable instructions for performing operations comprising:
receiving patient information associated with a patient;
receiving, via a graphical user interface, a request for health services for the patient;
in response to receiving the request for health services, applying a model to the patient information to select a first type of service of care from a plurality of types of service of care;
accessing a mesh application service architecture (MASA) to route traffic comprising a portion of the patient information to a server associated with the first type of service of care; and
generating, for display within the graphical user interface, health service information received from the server associated with the first type of service of care.
